(19) United States
(12) Patent Application Publication
(10) Pub. No.: US 2004/0242538 A1
(43) Pub. Date: Dec. 2, 2004

(54) Method to improve complexation efficacy and produce high-energy cylodextrincomplexes

(76) Inventors: Thorsteinn Loftsson, Reykjavik (IS); Mar Masson, Reykjavik (IS)

Correspondence Address: WHITE & CASE LLP, PATENT DEPARTMENT, 1155 AVENUE OF THE AMERICAS, NEW YORK, NY, 10036, US

(21) Appl. No.: 10/488,123

(22) PCT Filed: Aug. 26, 2002
(86) PCT No.: PCT/SE02/01519

(30) Foreign Application Priority Data
Aug. 27, 2001 (SE) 0102856-2

(51) Int. Cl. A61K 31/724

(52) U.S. Cl. 514/58

(57) ABSTRACT

This invention relates to a new method to improve the complexation efficacy of a basic active substance and a cyclodextrin using an acidic volatile substance. The invention further relates to a method to prepare high-energy complexes of a basic active substance and a cyclodextrin that form super-saturated solutions when dissolved. Also, the present invention relates to a pharmaceutical formulation comprising said complex and the use of such a formulation in therapy.

### FIELD OF THE INVENTION

This invention relates to a new method to improve the complexation efficacy of a basic active substance and a cyclodextrin using an acidic volatile substance to ionise the basic active substance. The acidic volatile substance is removed from the complex during drying and, optionally, during further prolonged drying resulting in a solid complex of the unionised basic active substance and cyclodextrin.

The invention further relates to a method to prepare high-energy complexes of a basic active substance and a cyclodextrin that form super-saturated solutions when dissolved. Also, the present invention relates to a pharmaceutical formulation comprising said complex and the use of such a formulation in therapy.

### BACKGROUND OF THE INVENTION

Cyclodextrins are known to form complexes with active substances such as drugs in aqueous solutions through a process by which water molecules in the central cavity of the cyclodextrin are replaced by a molecule of an active substance or by a lipophilic part of said molecule. Cyclodextrins may therefor be used in drug delivery to e.g. increase the bioavailabilty of a drug.

There are various methods to prepare complexes of a cyclodextrin and an active substance. The standard method to prepare such complexes is by addition of an excess amount of drug to an aqueous cyclodextrin solution. The suspension formed is equilibrated and then filtered or centrifuged to form a clear solution of the drug-cyclodextrin complex. Other methods to obtain drug-cyclodextrin complexes include the kneading method, co-precipitation and the grinding technique.

For a variety of reasons, including cost, production capacity and toxicity, the amount of cyclodextrin that can be used in a pharmaceutical formulation is limited. For example the ideal weight of a solid oral dosage form is between 50 and 500 mg. Even at high drug incorporation rates, one gram of a solid drug-cyclodextrin complex would only contain between 100 and 250 mg of the drug, assuming a drug with a molecular weight between 200 and 400 g/mol and a cyclodextrin with a molecular weight between 1200 and 1500 g/mol. Thus, even under the best conditions, cyclodextrin complexation will result in a 4- to 10-fold increase in the formulation bulk. This limits the use of cyclodextrins in solid dosage forms to relatively potent drugs, which possess very good complexing properties. Likewise, the maximum cyclodextrin concentration in isotonic solutions is between 20 and 25%. This means that for some drugs parenteral administration is not possible. Thus, there is a need to develop methods, whereby the efficiency of drug-cyclodextrin complexation is enhanced. By this way the present limitations of the use of cyclodextrins for pharmaceutical purposes can be reduced.

The equilibrium of the formed drug-cyclodextrin (D-CD) complex can be expressed as

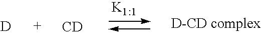

and

$${K}_{1 : 1} = \frac{[ D - CD ]}{[ D ] [ [ CD ]}$$

and the intrinsic solubility of the drug S₀=[D]. Increasing S₀ and/or K_{1:1} can increase the complexation efficacy. This can for example be done by increasing the solubility of the drug. Through pH adjustment, the drug can be ionised. This will increase the apparent intrinsic solubility (S₀) of the drug. Furthermore, the ionised drug can enhance the solubility of the cyclodextrin. This will result in a higher total solubility of both drug and cyclodextrin. Ionised drugs however form frequently less stable drug-cyclodextrin complexes than their unionised counterparts. This may result in a decrease of K_{1:1}. However, the increase in S₀ is often more than enough to compensate for the decrease in K_{1:1}, resulting in an overall enhanced complexation efficacy. Addition of low molecular weight acids, such as acetic acid, citric acid, malic acid or tartaric acid, to aqueous complexation media can enhance the solubilisation of basic drugs through an increase in S₀. In J. Mass. Spectrom., 30, 219-220, (1994) the possibility of enhancing complexation of various basic drugs through formation of drug-hydroxy acid-cyclodextrin ternary complexes or salts is described. During preparation of the ternary complexes the salt forming acid is not removed and, thus, still present in the complex. The hydroxy acids are non-volatile substances and high-energy complexes are thus not formed. These methods are suitable for preparing solutions with enhanced complexation efficacy. However, the preparation of solid complexes with high complexation efficacy continues to be a challenge. This is especially true when it is desired to prepare complexes of a drug in its base form rather than the corresponding conjugated acid (i.e. ionised) form.

### DETAILED DESCRIPTION OF THE INVENTION

A new method has now surprisingly been found in which the complexation efficacy of basic active substances and cyclodextrins is remarkably increased. The present invention relates to a method to improve the complexation efficacy of a basic active substance and a cyclodextrin using an acidic volatile substance, which is removed from the complex during drying to obtain a complex powder with a molar ratio of active substance:cyclodextrin:volatile substance of 1:1:0-0.50. The complex obtained may be further dried at elevated temperature and reduced pressure. Preferred molar ratios are 1:1:0-0.20. This new method is especially useful for poorly soluble or water-insoluble basic substances. It is also useful for poorly soluble cyclodextrins, such as β-cyclodextrin. The present invention provides for a method by which the basic active substance is ionised by addition of a volatile acid to an aqueous cyclodextrin solution. This cyclodextrin solution may be an aqueous acid solution or a cyclodextrin in a pure acid solution (i.e. a non-aqueous solution).

Ionisation of the drug molecule will increase the intrinsic solubility of the drug (S₀). This increase in S₀ will shift the equilibrium towards formation of the complex resulting in an enhanced complex formation. Enhanced complex formation will improve the complexation efficacy, which will reduce the formation bulk of the solid drug-cyclodextrin complexes. In general only some fraction of the cyclodextrin molecules form a complex and, thus, the complex powder produced contains a mixture of drug/cyclodextrin complexes and free cyclodextrin molecules. Enhanced complexation efficacy will reduce the amount of free cyclodextrin molecules in the complex powder. Further, the formation of a drug-cyclodextrin complex of the ionised drug will enhance the solubility of the cyclodextrin, e.g. through formation of water-soluble drug-cyclodextrin complexes. Increase in S₀ and increased solubility of the cyclodextrin and the drug-cyclodextrin complex will enhance the complexation efficacy.

A solid complex powder may be produced by precipitation of the solid drug-cyclodextrin complex from the solution, by evaporation of the solvent or through lyophilization or spray-drying. The solid drug-cyclodextrin complex will then be heated at elevated temperature, optionally under reduced pressure, to evaporate the volatile acid resulting in a drug-cyclodextrin complex of the unionised drug. (See FIG. 1)

One aspect of the present invention provides for a process for the preparation of a complex of a basic active substance and a cyclodextrin comprising the following steps;

a) adding of an acidic volatile substance to a solution of a cyclodextrin and a basic active substance,

b) optionally heating the solution,

c) optionally shaking the solution under cooling,

d) drying the solution,

e) sieving the complex and

f) optionally further drying the solid complex at elevated temperatures and reduced pressure.

The method of the present invention can be conducted at ambient or lower temperatures. Complexation efficacy of drug-cyclodextrin complexes can be increased by lowering the temperature due to the negative enthalpy of the stability constants (K_{c}) of the drug-cyclodextrin complexes. These lower temperatures make large-scale production more efficient.

Furthermore, cyclodextrins of limited water-solubility, such as natural cyclodextrins, especially β-cyclodextrin, can be solubilized using the method according to the present invention. This solubilization of the cyclodextrins will again promote the complexation and thus enhance the complexation efficacy. Consequently, this will result in a reduction of the production time of the drug-cyclodextrin complexes.

Also, the complexes can be formed under non-physiological conditions e.g. at very low pH. Drugs, which are unionisable under physiological conditions, can be prepared under these non-physiological conditions. Drug-cyclodextrin complexes of very weak bases may be prepared in anhydrous acetic acid. The volatile acid will be removed during the preparation of the solid drug-cyclodextrin complex. Thus, the new method of the present invention may be used for a wide variety of substances with very diverse chemical properties.

The present invention further provides for a method to prepare high-energy complexes of unionised basic active substances. These complexes increase the solubility of the drug temporarily. “High-energy” complex shall mean a complex, which is thermodynamically unstable. When the solid complex is dissolved in an aqueous solution it will form a super-saturated solution. Increasing the intrinsic solubility of the drug increases the complexation efficacy. After complexation, the intrinsic solubility of the drug is decreased by removing the volatile acid. However, the drug is stuck in the complex since the drug-cyclodextrin complex is already in the solid state. When dissolved, the decreased complexation efficacy enhances drug release from the complex. When super-saturated solutions are formed in the gastrointestinal tract, more rapid drug absorption will be observed. Thus, a “high-energy” drug complex results in an enhanced bioavailabilty after administration.

Active substances suitable to use in the method according to the present invention may be weak to strong basic substances. A non-limited list of active substances may include drugs such as (R)—N-[5-methyl-8-(4-methylpiperazin-1-yl)-1,2,3,4-tetrahydro-2-naphthyl]-4-morpholinobenzamide, tamoxifen, midazolam, alprazolam, diazepam, antibacterial agents such as oxazlidinones, proton pump inhibitors such as omeprazole, lansoprazole, pantoprazole, rabeprazole as well as their enantiomers such as for instance esomeprazole, and pharmaceutically acceptable salts of any of these compounds. (R)—N-[5-methyl-8-(4-methylpiperazin-1-yl)-1,2,3,4-tetrahydro-2-naphthyl]-4-morpholinobenzamide is described in WO 99/05134. This compound may be used for prevention and/or treatment of CNS disorders, especially 5-hydroxytryptamine mediated disorders as described in WO 99/05134, which is hereby incorporated by reference. Preferred active substances for the present invention are those that are insoluble or poorly soluble in water. Any class or subclass of cyclodextrin may be used in the method of the present invention. A non-limited list of cyclodextrins may include the natural cyclodextrins, α-cyclodextrin, β-cyclodextrin, γ-cyclodextrin or cyclodextrin derivatives such as hydroxypropyl-β-cyclodextrin, randomly methylated β-cyclodextrin, β-cyclodextrin sulfobutyl ether, maltosyl-β-cyclodextrin and hydroxypropyl-γ-cyclodextrin. Preferred cyclodextrins are α-cyclodextrin, β-cyclodextrin (βCD), γ-cyclodextrin and hydroxypropyl-β-cyclodextrin (HPβCD).

Appropriate acidic volatile substances suitable for complexation of basic active substances include, but are not limited to, acetic acid, formic acid, propionic acid and carbonic acid.

The term ‘insoluble’ shall mean that essentially less than 1% of the active substance is dissolved in an aqueous solution. The term ‘poorly soluble’ referres to a substance that dissolves very slowly in an aqueous solution, i.e. less than 10% being dissolved in a period of 1 hour.

The term “active substance” shall mean any chemical substance, preferably a pharmaceutically active substance e.g. a drug useful in therapy.

The term ‘volatile substance’ shall mean a compound having a vapor pressure between 0.2 and 1000 mmHg at 0° C.

The following examples are intended to illustrate, but in no way limit the scope of the invention.

### BRIEF DESCRIPTION OF THE DRAWINGS

FIG. 1. The process for a basic drug.

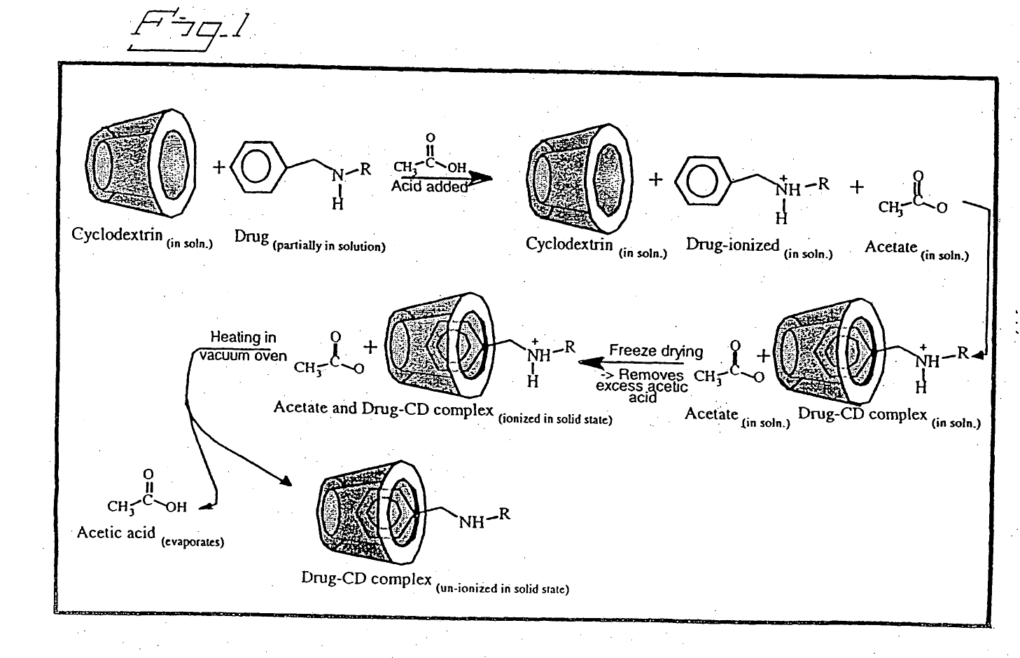

FIG. 2. Removal of acetic acid from the Basic drug A/HPβCD complexes in the vacuum oven at 88° C. and 0.13×10² Pa.

FIG. 3. Molar ratio of acetic acid left in the Basic drug A/βCD complex powder as the function of drying time in the vacuum oven at 88° C. and 0.13×10² Pa.

FIG. 4. Molar ratio of acetic acid left in the Basic drug B/βCD complex powder as the function of drying time in vacuum oven at 70° C. and 0.13×10² Pa.

FIG. 5. Dissolution profiles of the Basic drug A/HPβCD complexes.

FIG. 6. Dissolution profiles of the Basic drug A/βCD complexes dried for 4 days.

Abbreviations

| | |
|---|---|
| Basic drug A | (R)-N-[5-methyl-8-(4-methylpiperazin-1-yl)-1,2,3,4- |
| | tetrahydro-2-naphthyl]-4-morpholinobenzamide |
| Basic drug B | tamoxifen |
| CD | cyclodextrin |
| HPβCD | hydxoxypropyl-β-cyclodextrin |
| βCD | β-cyclodextrin |
| SD | standard deviation |

### METHOD

HPβCD or βCD was dissolved in distilled water (1 or 2% w/v solution) and an equimolar amount of the drug to be tested was added to the solution. Then 0, 1, 5, 10, 15, 20 or 50 mole equivalents of a volatile acid were pipetted into the solution. The solutions were subsequently heated in an autoclave for 20 minutes at a temperature between 100 and 130° C., preferably 121° C., to dissolve the drug (heating is not necessary if the drug is already dissolved by the volatile acid). The solutions were then placed in a shaker for 1 hour, during cooling, and subsequently lyophilized for 20-28 hours. The solid powder was sieved through a 300 μm sieve and 30-50 mg reserved for analysis. The rest of the powder was heated in a vacuum oven at 50-90° C. at 0.13×10² Pa for 1-14 days. The temperature maintained in the vacuum oven depended on the physicochemical properties of the drug.

Basic Drug/HPβCD

Basic drug A was tested together with HPβCD. Glacial acetic acid was used to dissolve the drug. The first complex that was prepared did not contain any acetic acid and this complex was used as a reference. When no acetic acid was in the solution with the drug and cyclodextrin, very little of the drug was dissolved and consequently, very little complexation occurred. All HPβCD had dissolved. 10 molar equivalents of acetic acid were needed to dissolve Basic drug A. If the solution was heated, then only 2-3 molar equivalents of acetic acid were needed to dissolve Basic drug A, i.e. 1 equimolar complex means that the molar ratio of drug:CD:acid is 1:1:1 and 10 equivalents complex means that the molar ratio of drug:CD:acid is 1:1:10.

Basic Drug/βCD

Two basic drugs were tested, i.e. Basic drug A and Basic drug B together with βCD. About 5 equivalents of acetic acid were needed to dissolve Basic drug A. If the solutions were heated, then only 2-3 equivalents of acetic acid were needed to dissolve Basic drug A. About the same amount of acetic acid was needed to dissolve Basic drug B.

Example of Complexes

TABLE 1
Basic drug A/HPβCD complex prepared with 10 equivalents of acetic
acid (using 1% w/v HPβCD solution).

| Drug | Basic drug A | |
|---|---|---|
| Mol weight of drug | 449 | |
| HPβCD weighed | 500 | mg |
| Drug weighed | 160.4 | mg |
| Glacial acetic acid pipetted into | 204 | μl |
| solution | | |
| | Add water to 50 | ml |
| Measured pH of solution before | 3.71 | |
| heating | | |
| Measured pH of solution after heating | 3.74 | |

TABLE 2
Basic drug A and B/βCD complexes prepared with 5 equivalents
of acetic acid (using 2% w/v βCD solution).

| Drug | Basic drug A | | Basic drug B | |
|---|---|---|---|---|
| Mol weight of drug | 449 | | 371.5 | |
| βCD weighed | 1000 | mg | 500 | mg |
| Drug weighed | 396.5 | mg | 163.7 | mg |
| Glacial acetic acid | 252 | μl | 126 | μl |
| pipetted | | | | |
| into solution | | | | |
| | Add water to 50 | ml | Add water to 25 | ml |
| Measured pH of solution | 3.82 | | 3.80 | |
| before heating | | | | |
| Measured pH of solution | 4.03 | | 3.97 | |
| after heating | | | | |

All solutions were lyophilized, then the obtained complex was sieved through a 300 μm sieve and heated in a vacuum oven at 60-90° C. for several days, depending on the drug being investigated. Samples were taken each day from the oven for acetic acid determination.

Quantitative Determination of Drug in Solid Freeze-Dried Complexes.

The amount of drug per gram sample was determined on a high performance liquid chromatographic (HPLC) component system, consisting of ConstaMetric 3200 solvent delivery system operated at 1.5 ml/min, a SpectroMonitor 3200 UV/VIS variable-wavelength detector, a Merck-Hitachi AS-2000A autosampler, Merck Hitachi D-2500 Chromato-Integrator and a Phenomex ODS 5 μm (150×4.6 mm) column.

Selected Basic drug A complexes were analysed after heating in the vacuum oven at 90° C. for several days. No decrease in the drug concentration could be observed. Basic drug A appeared to be stable during heating in the vacuum oven. Selected Basic drug B complexes were analysed after heating in the vacuum oven at 70° C. for several days. Basic drug B was less stable during heating, and degradation products were observed on the HPLC.

Determination of Acetic Acid.

Indirect measurement of acetic acid were performed by measuring the increase in light absorbance due to NADH formation.

Acetic acid (acetate) is converted, in the presence of the enzyme acetyl-CoA synthetase (ACS) with ATP and CoA, to acetyl CoA:

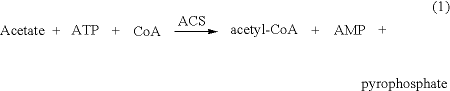

Acetyl CoA reacts with oxaloacetate to form citrate in the presence of citrate synthase (CS):

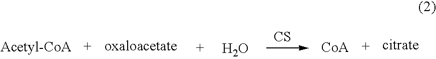

The oxaloacetate required for reaction (2) is formed from L-malate and NAD in the presence of L-malate dehydrogenase (L-MDH) (3). In this reaction NAD is reduced to NADH:

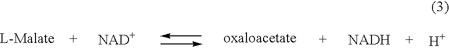

The detection limit is 0.150 μg/ml.

Removal of Acetic Acid—Rate Profile.

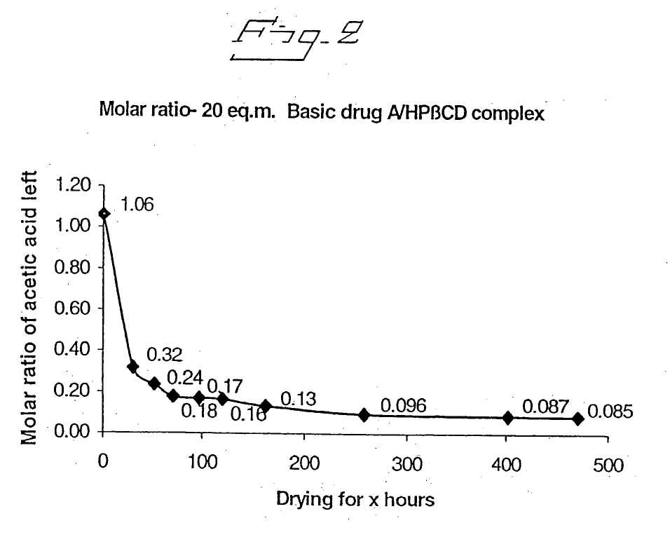

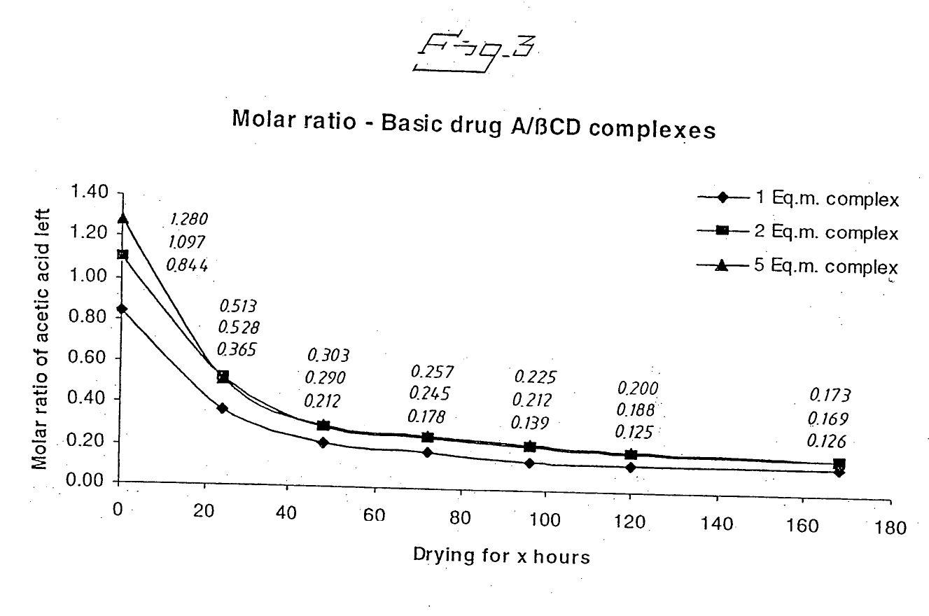

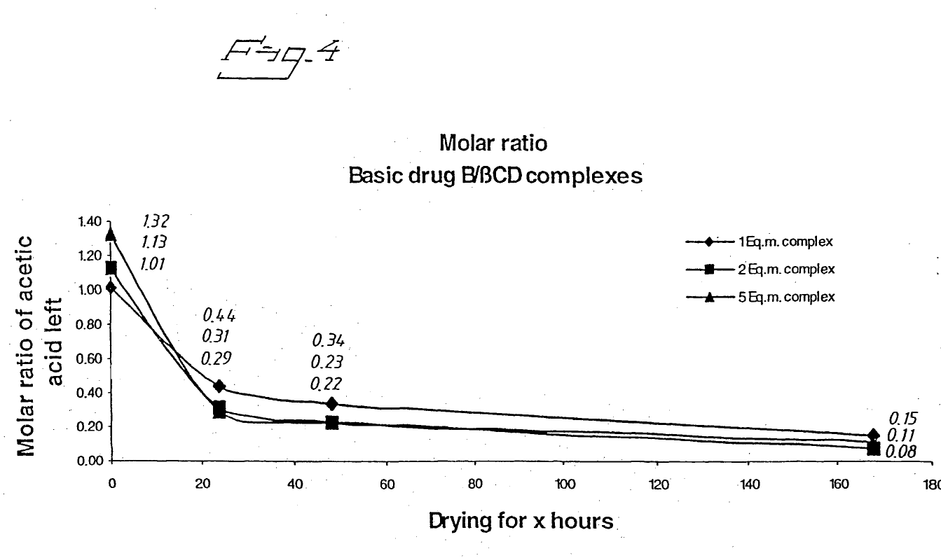

20 molar equivalents complex of Basic drug A/HPβCD was prepared and 30-40 mg were reserved for acetic acid analyses. The solid complex was placed in the vacuum oven at 88° C. at 0.13×10² Pa. At one-day intervals (later at 4-5 day intervals) a small sample was collected and kept for acetic acid analyses. After three weeks all the samples were analysed by the enzymatic technique described above. FIG. 2 shows that normal lyophilization removes about 19 equivalents of acetic acid. That is normal because it is assumed that 19 equivalents of acetic acid are unbound and 1 equimolar is bound to the drug (drug-ionisation). Heating in the vacuum oven results in an effective removal of acetic acid during the first 20-30 hours. A molar ratio of 0.085 acetic acid means that the molar ratio between Basic drug A:HPβCD:acetic acid is 1:1:0.085. One gram of this complex then contains 0.76 gram of HPβCD, 0.24 gram of Basic drug A and only 0.0027 gram of acetic acid (0.27% w/w). Similar curves were obtained when the removal of acetic acid from Basic drug A/βCD complexes were plotted. These complexes were prepared by using 1, 2 and 5 equal molar of acetic acid respectively, heated for 20 minutes at 121° C. in autoclave, cooled and shaken for one hour and subsequently freeze-dried for 24 hours. The solid complex powder was then placed in a vacuum oven at 88° C. and 0.13×10² Pa. A small sample was taken at one-day intervals and kept for acetic acid analyses. The results are shown in FIG. 3. Rate profiles for the removal of acetic acid from Basic drug B/βCD complexes are less accurate because there were some Basic drug B degradation products that interfered with the enzymatic technique. However, the curves in FIG. 4 show the same trend as in FIGS. 2 and 3. The complexes were prepared and analysed as described above.

Formation of High-Energy Complexes.

To investigate this special property of the complexes, 50 mg of complex containing the basic drug was dissolved in 25 ml of phosphate buffer 0.1 M, pH 7.4. 100 μl samples were taken from this solution and measured for 1-96 hours depending on the complex being investigated. A complex prepared without acid was used as a reference to show this special high-energy effect. Solid drug particles with and without cyclodextrin were also weighed and placed in the buffer to show the difference in solubility between samples of the drug alone, drug and cyclodextrin in a standard mixture, and drug in complex with cyclodextrin.

High-Energy Basic Drug A/HPβCD Complexes

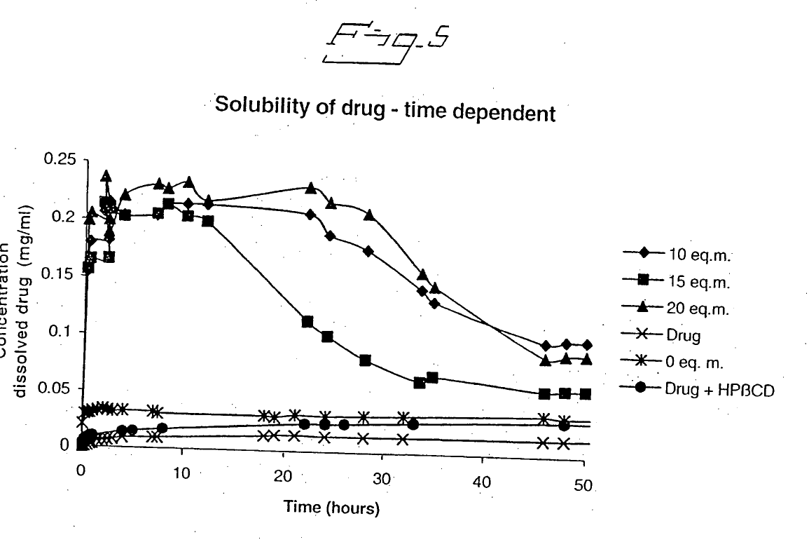

The solubility of the drug from four different complexes, prepared with 0, 10, 15 and 20 equivalents of acetic acid, was measured. The solid complexes were heated in a vacuum oven for 4 days at 88° C. and 0.13×10² Pa. Each complex contained about 0.2 equivalents of acetic acid. A 50 mg sample of each complex was dissolved in 25 ml of the buffer. Each experiment was repeated 3 times and the results shown are the mean values±standard deviation (SD). Basic drug A/HPβCD complex prepared without acetic acid (0 eq. complex) was used as a reference to show the effect of the acetic acid. 12 mg of solid Basic drug A particles were also dissolved in 50 ml of the buffer with or without 38 mg of HPβCD to show the effect of complexing. The results are shown in FIG. 5. The solubility of the drug is about 0.20-0.25 mg/ml (from complexes containing acetic acid) for the first 10-20 hours and then the solubility slowly decreases to about 0.05 mg/ml. The intrinsic solubility of the drug is 0.034 mg/ml. The solubility of about 0.05 mg/ml is due to the presence of cyclodextrin in the aqueous medium. FIG. 5 shows that there is a significant difference in drug-solubility between a complex made with (10, 15, 20 eq.) and without (0 eq.) acetic acid for the first 10-20 hours. FIG. 5 also shows that there is a significant difference in drug-solubility between 0 eq. complex and a mixture containing drug and cyclodextrin. The complexes prepared by the method according to the present invention result in formation of supersaturated drug solution whereas complexes prepared by a conventional method only gave saturated solutions.

High-Energy Basic Drug A/βCD Complexes

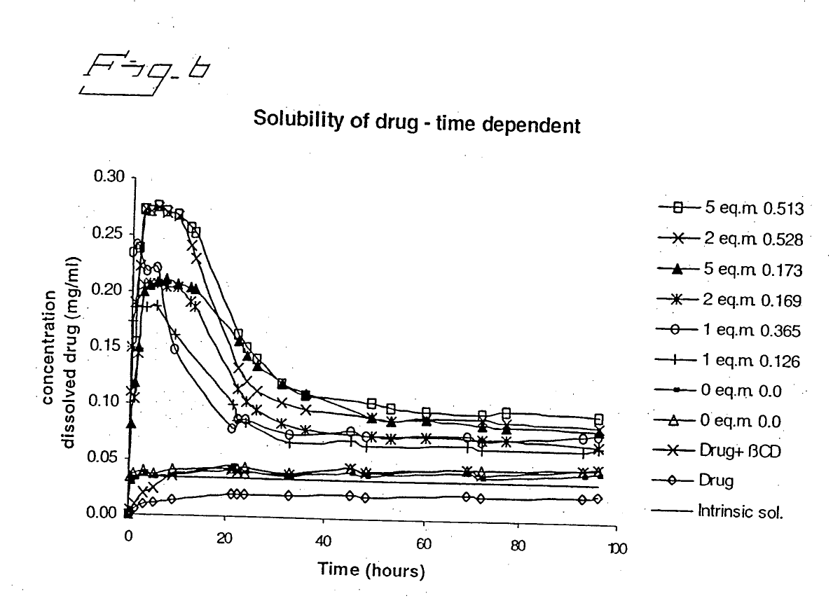

The solubility of the drug from five different complexes, prepared with 0, 1, 2, 5 and 10 equivalents of acetic acid, was measured. The 10 equal molar complexes were dried in a vacuum oven for 4 days at 60° C. and 0.13×10² Pa. Half of the 0, 1, 2 and 5 equal molar complexes were heated for 1 day at 88° C. and 0.13×10² Pa and the other half was heated for 7 days at 88° C. and 0.13×10² Pa. The acetic acid residue left in the solid complex was measured and the results are shown in FIG. 6. A 50 mg sample of each complex was dissolved in 25 ml of the buffer. Each experiment was repeated three times and the results shown are the mean values±SD. Samples were taken and measured for 96 hours. Basic drug A/βCD complex prepared without acetic acid was used as a reference to show the effect of the acetic acid. 14 mg of solid Basic drug A particles were also dissolved in 50 ml of the buffer with or without 36 mg of βCD to show the effect of complexing. The legend name describes how much acetic acid was used when preparing the complex and how much acetic acid is left after drying. The drug-solubility peak is from 0.18-0.32 mg/ml (for complexes containing acetic acid) for the first 10-20 hours and then the solubility slowly decreases to about 0.06-0.10 mg/ml depending on how much acetic acid was left in the solid complex. FIG. 6 shows that there is a significant difference in drug-solubility between the complex made with (1, 2, 5 and 10 eq.) and without (0 eq.) acetic acid for the first 10-20 hours.

Pharmaceutical Formulation.

According to one aspect of the present invention there is provided a pharmaceutical formulation comprising a drug-cyclodextrin complex prepared with the method of the invention that may be used for the manufacture of a medicament. This pharmaceutical formulation may be used in therapy. The present invention relates to a pharmaceutical formulation that may be used for the treatment and/or prevention of different kind of disorders and medical disturbances. Pharmaceutical formulations for the treatment and/or prevention of 5-hydroxytryptamine mediated disorders are of particular interest. The pharmaceutical formulation, optionally in association with adjuvants, diluents, excipients and/or carriers, may be prepared in a conventional manner using conventional excipients.

The pharmaceutical formulation may be in a form suitable for oral administration, for example as a tablet, pill, syrup, powder, granule or capsule, for parenteral injection (including intravenous, subcutaneous, intramuscular, intravascular or infusion) as a sterile solution, suspension or emulsion, for topical administration as an ointment, patch or cream or for rectal administration as a suppository.

Suitable daily doses of the active ingredients may vary within a wide range and will depend on various factors such as the relevant therapy, the route of administration, the age, weight and sex of the mammal. Suitable daily doses may be determined by a physician.

1. A method to improve the complexation efficacy of a basic active substance and a cyclodextrin using an acidic volatile substance, whereby the volatile substance is removed from the complex during drying to obtain a complex powder with a molar ratio of active substance:cyclodextrin:volatile substance of 1:1:0-0.50.

2. The method according to claim 1, whereby the complex is further dried at elevated temperature and reduced pressure.

3. The method according to any one of claims 1 and 2, whereby the molar ratio of active substance:cyclodextrin:volatile substance is 1:1:0-0.20.

4. The method according to any one of claims 1 to 3, whereby the drying method is lyophilization.

5. The method according to any one of claims 1 to 3, whereby the drying method is spray-drying.

6. The method according to any one of claims 1 to 5, whereby the acidic volatile substance is selected from the group of acetic acid, formic acid, propionic acid and carbonic acid.

7. The method according to any one of claims 1 to 6, whereby the cyclodextrin is selected from the group of α-cyclodextrin, β-cyclodextrin, γ-cyclodextrin, hydroxypropyl-β-cyclodextrin, randomly methylated β-cyclodextrin, β-cyclodextrin sulfobutyl ether, maltosyl-β-cyclodextrin and hydroxypropyl-γ-cyclodextrin.

8. The method according to claim 7, whereby the cyclodextrin is β-cyclodextrin or hydroxypropyl-β-cyclodextrin.

9. The method according to any one of claims 1 to 8, whereby the basic active substance is a substance insoluble or poorly soluble in water.

10. The method according to any one of claims 1 to 8, whereby the basic active substance is selected from the group of (R)—N-[5-methyl-8-(4-methylpiperazin-1-yl)-1,2,3,4-tetrahydro-2-naphthyl]-4-morpholinobenzamide and tamoxifen.

11. The method according to any one of claims 1 to 8, whereby the basic active substance is selected from the group of antibacterial agents such as oxazlidinones, proton pump inhibitors such as omeprazole, lansoprazole, pantoprazole, rabeprazole as well as their enantiomers such as esomeprazole,and pharmaceutically acceptable salts of any of these substances.

12. A process for the preparation of a complex of a basic active substance and a cyclodextrin according to any one of claims 1 to 11, wherein the process comprises the following steps;
a) adding of an acidic volatile substance to a solution of a cyclodextrin and a basic active substance,
b) optionally heating the solution,
c) optionally shaken the solution under cooling,
d) drying the solution,
e) optionally sieving the complex and
f) optionally further drying the solid complex at elevated temperatures and reduced pressure.

13. The process according to claim 12, whereby the solution in step b) is heated for 20 minutes at a temperature between 100 and 130° C.

14. The process according to claim 12, whereby the solution in step c) is shaken for 1 hour.

15. The process according to claim 12, whereby the complex in step f) is heated under vacuum at a temperature between 50 to 90° C.

16. Use of a complex prepared according to the method of any one of claims 1 to 11 for the manufacture of a pharmaceutical formulation.

17. A pharmaceutical formulation comprising the complex prepared by the method according to any one of claims 1 to 11, optionally in association with adjuvants, diluents, excipients and/or carriers.

18. The pharmaceutical formulation according to claim 17 for use in therapy.

19. The pharmaceutical formulation according to claim 17 for the treatment and/or prevention of 5-hydroxytryptamine mediated disorders.

20. Use of a pharmaceutical formulation according to any one of claims 17 to 19 for the manufacture of a medicament.

21. High-energy complexes comprising an unionised basic active substance, a cyclodextrin and an acidic volatile substance in a molar ratio of 1:1:0-0.50, prepared by the method according to any one of claims 1 to 11 that form a supersaturated solution when dissolved in an aqueous solution.

22. The complex according to claim 21, wherein the molar ratio of active substance:cyclodextrin:volatile substance is 1:1:0-0.20.
